(19) United States
(12) Patent Application Publication
(10) Pub. No.: US 2002/0065683 A1
(43) Pub. Date: May 30, 2002

(54) System and methods for providing pharmaceutical product information

(76) Inventors: Quang X. Pham, Mission Viejo, CA (US); David A. Ramsay, San Pedro, CA (US); Alan P. Heim, Vista, CA (US); Michael G. Wells, Skippack, PA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/917,253

(22) Filed: Jul. 27, 2001

Related U.S. Application Data
(60) Provisional application No. 60/221,869, filed on Jul. 28, 2000.

(51) Int. Cl. G06F 17/60; G09B 3/00; G09B 7/00

(52) U.S. Cl. 705/2; 705/10; 705/14; 434/322

(57) ABSTRACT

A system provides a web site through which physicians can access information about multiple drugs provided by multiple drug companies. A user is authenticated as being a registered physician before being allowed access to the system. The system provides an interactive on-line detail or marketing presentation of a drug. The interactive detail provides information about a drug in addition to requesting and receiving responses or input from the user participating in the interactive detail. Questions and challenges are presented to the user to reinforce concepts, such as a drug's mechanism of action, that are presented to the user during the detail. Users' responses to interactive details are accumulated and provided to the respective drug companies that sponsor the details. As an incentive, the system provides an honorarium or gift to targeted users upon completion of interactive presentations.

### RELATED APPLICATIONS

This application claims the benefit of U.S. Provisional Application No. 60/221,869, filed on Jul. 28, 2000, which is hereby incorporated by reference.

### BACKGROUND OF THE INVENTION

1. Field of the Invention

This invention relates generally to systems and methods through which companies market products to and interact with clients, and, more particularly, the invention relates to systems and methods through which pharmaceutical companies can provide pharmaceutical product information to and interact with physicians.

2. Description of the Related Art

Pharmaceutical companies have employed substantially the same approach to marketing and “detailing” (making sales calls) to physicians for over 40 years. A pharmaceutical sales representative travels to the doctor's office, announces himself to the office receptionist or to the nurse at a hospital, and waits to see a physician to make his sales presentation. If the physician has time between seeing patients and other professional duties, the physician meets with the sales rep, and the sales rep provides the physician with promotional brochures and drug samples. In addition, pharmaceutical companies spend millions of dollars each year on meals and events in an attempt to create physician access for their representatives.

Detailing to physicians has been the primary promotion vehicle for pharmaceutical companies for over 40 years. While the number of pharmaceutical sales representatives, as tracked by Scoft-Levin, has climbed 61% in the past 5 years, details to physicians have been relatively flat (up 9%) over the same time period. There are currently in excess of 80,000 drug representatives in the market, which includes over 70,000 full-time representatives and 10,000+ part-time/contract sales representatives. Current industry reports indicate that this number is still increasing despite the apparent saturation disclosed by the Scott-Levin report. While the AMA reports 650,000 practicing physicians, the promotional focus of the pharmaceutical industry is on the top two deciles. This means that 80,000+ sales representatives (and increasing) are calling on approximately 130,000 physicians, nearly a 1 to 1 ratio.

The pharmaceutical industry spent $15 billion in 2000 on promotional efforts targeting physicians including traditional detailing. However, the Health Care Strategies group released a study in December 1999 indicating that 87% of details last less than 2 minutes, which is insufficient to deliver a complete detail. The study also found that 43% of sales calls do not result in the sales representative speaking with a doctor. To compound this issue, the industry pays between $150-$250 for each of these incomplete details according to an equity research report from Credit Suisse First Boston.

Given this dynamic, the pharmaceutical industry is actively seeking new ways to drive incremental prescribing. Evidence for this statement is illustrated by the $2 billion investment in Direct-to-Consumer (DTC) advertising in 2000. This amount represents a 100% increase over 1999 (Source: Advertising Age). This trend indicates that the pharmaceutical industry will embrace a new channel if the new channel can be used effectively to produce measurable return on investment. While DTC advertising can be very effective, many pharmaceutical brands are not well suited for the DTC channel due to their safety profile. Furthermore, DTC advertising requires a substantial investment in order to create the desired effect. Thus, DTC advertising alone will not remedy the marketing challenges that face the drug industry and achieve increased incremental prescribing goals.

Yet another important trend in the pharmaceutical industry is the current pipeline for prescription medications. According to the Pharmaceutical Manufacturers Association (PhRMA), there are thousands of compounds in development, including 600 compounds for the treatment of aging and 369 biotechnology medicines in testing. The pipeline of future medicines compounded with the hundreds of pharmaceutical brands that are aggressively marketed by drug companies today cannot be supported by the current sales infrastructure.

Physicians nowadays are busier than ever. Due to managed care influence, physicians have assumed greater patient loads, and gaining access to physicians is extremely difficult for pharmaceutical representatives. Doctors generally appreciate the information and services provided by these representatives; however, representatives require physician time when it is least available due to patient loads and administrative pressures.

### SUMMARY OF THE INVENTION

In accordance with one embodiment, a physician is able to use any personal computer with web access to obtain information about drugs from a licensed database, to contact a pharmaceutical company/manufacturer to report any adverse drug reaction, to request and schedule an appointment with a sales representative, to request samples from a drug manufacturer, and to keep abreast of clinical trials, all from a centralized hub. In accordance with one embodiment, the invention enables drug companies to more efficiently and effectively market products to doctors. One embodiment enables the drug industry to provide additional sales support for marketing products to targeted physicians. One embodiment enables drug companies to leverage their existing sales and Internet investments to enable the aforementioned benefits without interrupting the busy schedules of physicians.

In accordance with one embodiment, a system provides a centralized on-line location from which physicians can access information about multiple drugs provided by multiple drug companies. Any of the multiple drug companies can contact any of multiple physicians through the system to provide, for example, urgent information about a drug. A user can be authenticated as being a registered physician before being allowed access to the system. The system preferably provides an interactive on-line detail or marketing presentation of a drug through a computer interface. The interactive detail provides information about the drug in addition to requesting and receiving responses or input from the user participating in the interactive detail. Physicians' responses to interactive on-line details are accumulated and provided to the respective drug companies that sponsor the details. The responses and/or other data relating the doctors' use of the system is preferably provided to the respective drug companies automatically by integrating the system with drug company customer relationship management (CRM) systems. The system enables sales representatives to set up home pages or web sites that are hosted by the system. A sales representative can provide a busy doctor with a business card that has a uniform resource locator (URL) through which the doctor can reach the representative's home page on the system. Through the home page, the doctor can link to interactive details, access any information the sales representative may want to present, or communicate with the representative through on-line facilities such as e-mail or HTML forms. The system provides a call center partnership through which multiple drug companies can outsource typical call center activities to increase efficiency and availability. The system provides a systematic segmentation scheme wherein physicians are placed into segments based upon available contact information. A sequence of communications through which physicians in each segment can be contacted is provided based upon available communication channels for the respective segment. Segments can also or alternatively be based upon communication frequency, timing, or information that is to be presented to the physicians in a segment. The system provides honoraria (gifts) in response to physicians' completion of interactive details. The honoraria can be offered only to certain targeted physicians or the honoraria can be offered to all physicians.

One embodiment of the invention is a system for providing pharmaceutical information to physicians. The system includes a physician authentication module configured to authenticate that a user is a registered physician. The system also includes a presentation hosting module configured to present a plurality of interactive presentations to users that have been authenticated by the physician authentication module, wherein each presentation is related to a prescription drug. The system also includes a data accumulation module configured to accumulate user responses to the interactive presentations.

One embodiment of the invention is a method for providing information related to pharmaceuticals to physicians. The method includes inviting a user to access a system configured to provide the information related to pharmaceuticals. The method also includes authenticating that the user is a physician. The method also includes presenting information related to a pharmaceutical to the user. The method also includes prompting the user to provide input confirming the user's comprehension of at least a portion of the presented information, and in response, receiving input provided by the user. The method can also include providing an honorarium to the user in response to receipt of the input.

One embodiment of the invention is a method including hosting interactive presentations related to prescription pharmaceuticals, accumulating user responses to the interactive presentations, presenting data related to accumulated user responses to drug companies sponsoring the interactive presentations, and providing honoraria to users in exchange for participation in the interactive presentations.

### BRIEF DESCRIPTION OF THE DRAWINGS

FIG. 1 illustrates a schematic overview of the functioning of a system in accordance with one embodiment.

FIG. 2 illustrates a schematic overview of certain functional modules of the system in accordance with one embodiment.

FIG. 3 illustrates a method in accordance with one embodiment of the invention.

### DETAILED DESCRIPTION OF THE INVENTION

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present invention. The present invention, however, may be practiced without the specific details or with certain alternative equivalent components and methods to those described herein. In other instances, well-known components and methods have not been described in detail so as not to unnecessarily obscure aspects of the present invention.

I. Overview

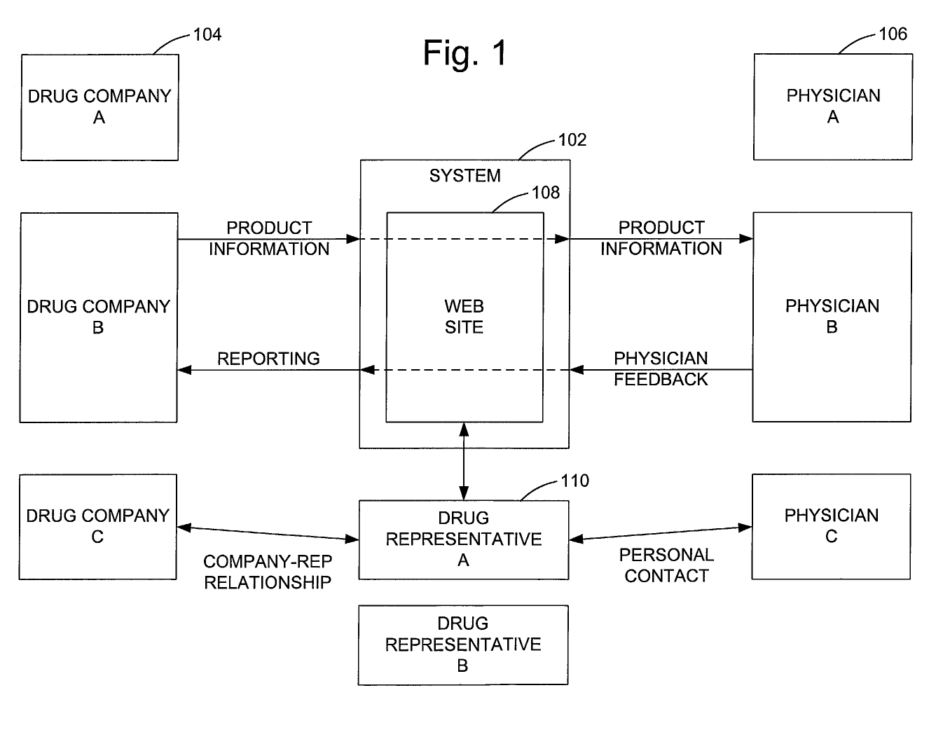

FIG. 1 illustrates an overview of the functioning of a system 102 in accordance with one embodiment. The system 102 serves as an intermediary between pharmaceutical (drug) companies 104 and physicians 106. The system 102 also facilitates the job of drug company sales representatives 110 in marketing prescription drugs to physicians 106 who might then prescribe the drugs. In the illustrated embodiment, three drug companies 104, three physicians 106, and two drug representatives 110 are shown for illustrative purposes. As will be understood by one skilled in the art, the system 102 can be configured to interact with any number of drug companies, such as tens or hundreds, any number of physicians, such as thousands or tens of thousands, and any number of representatives.

The system 102 serves as a central hub or location through which multiple physicians can receive product information from and provide feedback to multiple drug companies 104. The system 102 is preferably accessed by physicians 106 through a web site 108 that is hosted by the system 102.

Product information can be presented to physicians in the form of interactive details (i.e., sales presentations), which provide the information about a product in addition to interactively engaging the participating physician. The interactive details can increase the effectiveness of the details and also enable information to be obtained from physicians.

The system 102 can also be configured to compile and process feedback obtained from the physicians and to report this information to the drug companies 104. The system 102 can also be configured to provide drug representatives 110 an alternative line of communication with physicians and an alternative mechanism for presentation of information to physicians.

The system 102 is preferably hosted by a configuration of routers, firewalls, load directors and content engines, web servers and database servers. The database servers are preferably clustered, and a database is preferably stored on a RAID device. The database is also preferably backed up to tape on-line. The entire configuration is preferably completely redundant to ensure 24×7 operation. The web site 108 is preferably monitored continuously by a Linux server. In the unlikely event of the web site 108 going down, the Linux server preferably sends email to or pages responsible individuals. The hardware configuration of the system 102 will not be described in additional detail as the invention can be enabled through various hardware implementations as will be understood by one skilled in the art.

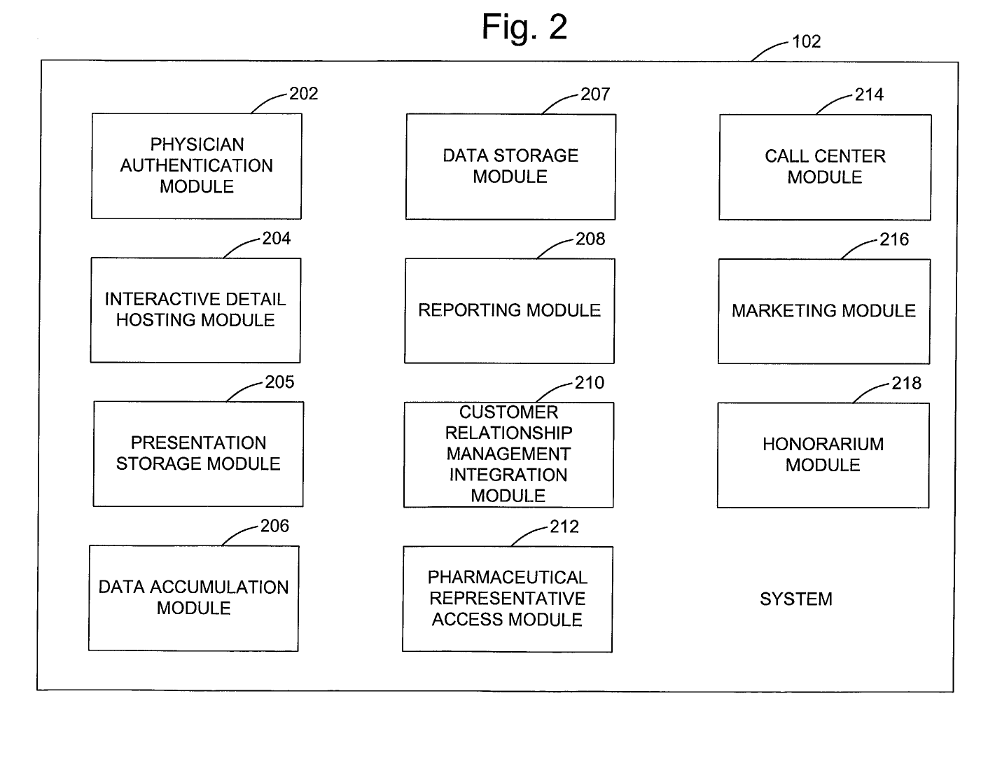

FIG. 2 illustrates an overview of certain functional modules of the system 102 in accordance with one embodiment. The system 102 preferably includes several modules that perform various functions.

Some or all of the modules can be embodied as software modules that are executed by computer hardware of the system 102. As will be understood by one skilled in the art, these modules can but need not be embodied as separate sections of computer code. The functionality of various modules can be embodied together in one or more code sections.

Some modules can be embodied partly or wholly through human participation, such as by setting up a team of employees to perform a certain function. In some instances, modules can be embodied using human participation in addition to, in conjunction with, or instead of computer code executing on hardware. The human participation may involve, for example, human analysis or interpretation of data, human use of the computer code, or human interaction with physicians that use the system.

A physician authentication module 202 is preferably configured to authenticate users as being registered physicians before allowing access to the system 102.

An interactive detail (presentation) hosting module 204 presents interactive details to physicians. The interactive details are preferably provided on-line through the web site 108 and are viewed through a web browser. Each interactive detail preferably provides information about a drug in addition to requesting and receiving responses or input from the physician participating in the interactive detail. In one embodiment, the hosting module 204 includes a presentation storage module 205 which stores the interactive presentations.

A data accumulation module 206 accumulates data from physicians that use the system. The accumulated data can include, for example, responses to interactive details, personal or contact information for physicians, and logs of each physician's use of the system. Some data can be accumulated for the use of all drug companies 104 that use the system 102. Other data can be accumulated for the specific use of certain drug companies, such as responses to interactive details, which can be accumulated for the use of the drug company sponsoring the detail.

A data storage module 207 is used to store data and is preferably embodied as a database. The data can include data accumulated by the data accumulation module and any other data that is collected by, used by, or provided by the system 102.

A reporting module 208 provides access to certain data stored in the data storage module 207 to the drug companies 104. The accessed data can include, for example, data accumulated by the data accumulation module 206, such as, for example, physician contact information or responses to interactive details. In one embodiment, the reporting module 208 can support a web page or web site through which a drug company can access the data.

A customer relationship management system integration module 210 provides an integrated connection between the system 102 and separate customer relationship management systems of individual drug companies. Typically, drug companies have their own software and systems for managing customer contact and relationship information. These systems can include contact information for physicians and other information that can assist salespeople in maintaining customer relationships. The customer relationship management system integration module 210 provides an automated connection through which some or all of the data accumulated by the data accumulation module 206 is automatically made available through drug companies' customer relationship management systems.

A pharmaceutical representative access module 212 hosts home pages or web sites of drug representatives 110. In accordance with one embodiment, a drug representative can provide a busy doctor with a business card that has a URL through which the doctor can reach the representative's home page on the system. Through the home page, the doctor can link to interactive details, access any information the sales representative may want to present, or communicate with the representative through on-line facilities such as e-mail or HTML forms.

A call center module 214 supports a telephone number that physicians can call to obtain information or make requests related to pharmaceuticals from one or more drug companies 104. The call center module 214 preferably supports most or all of the functionality that is typically provided by a call center of any individual drug company 104. The call center module 214, however, is preferably configured to provide these services for multiple drugs and for multiple drug companies. The call center module 214 can provide a single point of contact for several drugs rather than requiring physicians to find the proper number to call for information about a particular drug. In one embodiment, the call center module 214 can be implemented through human support, preferably with the assistance of computer code and hardware. Alternatively or additionally, the call center module 214 can be configured with an automated voice response system with or without the need for human support.

A marketing module 216 provides customized and comprehensive marketing plans. A systematic segmentation scheme places physicians into segments based upon available contact information or other information. The contact information can be obtained from or stored in the data storage module 207. The segmentation scheme preferably establishes a sequence of communications through which physicians in each segment can be contacted. The sequence is preferably based upon available communication channels for the respective segment. Segments can also or alternatively be based upon communication frequency, timing, or information that is to be presented to the physicians in a segment.

An honorarium module 218 is configured to provide a physician honorarium (gift or reimbursement) to targeted physicians based upon participation in interactive details. The honorarium can increase physician participation in the detail. In one embodiment, the receipt of the honorarium can be made contingent upon completion of responses requested by interactive details. In one embodiment, an honorarium fulfillment transaction is completed electronically through the Internet by providing a credit to a physician at a participating Internet-accessible vendor.

II. System Components

A. Physician Authentication Module

Physicians are directed through a verification process as part of the registration on the web site. The physician authentication module 202 preferably prompts the user to enter the following information: Email address, First and Last Name, State License Number, Licensing State, Drug Enforcement Agency (DEA) Number, Birth Date and Medical School.

The physician authentication module 202 is preferably a rules-based system that relies on three different physician verification databases—American Medical Association (AMA) database, State License Database and the Drug Enforcement Agency (DEA) Database. The following checks are preferably applied to the information entered by the user:

1. State License—Check1

a. Strip Initial Zeros from user State License Number

b. Strip zeros after initial alpha characters from user State License Number

c. Check State License Number, State, First and Last Name against State License Database

2. State License—Check 2

a. Strip initial alpha characters from user State License Number

b. Check State License Number, State, First and Last Name against State License Database

3. AMA—Check 1

a. Check Medical Education (ME) number from State License Database, Medical School, Date of Birth, First and Last Name against AMA Database

4. AMA—Check 2

a. Check ME number from State License Database, Medical School, Date of Birth against AMA Database

5. AMA—Check 3

a. Check Medical School, Date of Birth, First and Last Name against AMA Database

6. DEA—Check 1

a. Check DEA number, First and Last Name against the DEA Database

In accordance with one embodiment the following verification rules are applied:

1. Any matches found in at least two of the three physician verification databases result in the user being flagged as “AutoVerified”

2. Matches found in fewer than two of the physician verification databases result in the user being flagged as “Manual Verification Required”

3. If any particular database check (e.g. AMA—Check 1) succeeds, then the subsequent checks in the same database (e.g. AMA—Checks 2 & 3) can be skipped.

4. A single-record match in the AMA Database yields a positive ME number match. A multi-record match or no match in the AMA Database results in the user being flagged with “Manual ME Number Resolution”

5. For name matching, the first non-breaking string of alphabetic characters (the hyphen “-” is also included) is extracted from the user input and used in a leading wildcard search.

The following is an example of an application of the above rules. If the user enters “Jane R.” in the First Name field and “Smith-Jones, MD” in the Last Name field, then the database is searched for first names beginning “jane . . . ”, and last names beginning “smith . . . ” and containing the string “ . . . jones.” somewhere thereafter. The “R.” in the First Name field and the “,MD” in the Last Name field are ignored.

In accordance with one embodiment, users who are automatically verified are able to register and access the entire site. A user may enter information that has been used by another member as a result of mistyping verification data, another using the user's verification information, or the same user registering multiple times. If the user enters information that has been used by another existing member, the user is allowed access to the site for a single session only.

Users that are classified for manual resolution are checked manually against the three physician verification databases. In this case, honoraria/incentives and registration at the web site can be delayed until after certain manual checks have been completed. If the manual checks fail, the user is preferably sent an email requesting him to fax licensing information to the system 102.

The AMA, State License and DEA databases are preferably updated each month with information gathered from the AMA and State License Boards.

All verification information is preferably encrypted using SSL technology. This prevents the information from being intercepted and accessed while it travels across the Internet. The databases on the web site are preferably hosted by the data storage module 207 on database servers that are located behind multiple firewalls and are on a separate VLAN (virtual local area network) that is not on the Internet.

As will be understood by one skilled in the art, other verification methods can be used in the alternative.

B. Interactive Detail Hosting Module

The interactive detail hosting module 204 preferably presents on-line interactive details through the website 108. The hosting module 204 can include or communicate with the presentation storage module 205 upon which the interactive presentations are stored. The presentation storage module 205 can include one or more disk drives, a server, or a storage array.

The interactive details can be produced in different formats, such as, for example:

1. Macromedia Flash format, containing audio content, for a rich media version. This content is suited for high connection speeds; and

2. HTML format, containing gif and/or jpeg images. This content is suited for slow connection speeds.

Image optimization tools can be used to optimize content for low bandwidth.

An on-line interactive detail is preferably a concise, high-impact, interactive detail that takes place online. An interactive detail can include, for example, a traditional product detail or it can describe any physician-targeted program. An interactive detail preferably leverages existing marketing investments, such as a consistent marketing message and current core visual aid. An interactive detail can add animation and interactivity to existing sales aids. The interactive details are preferably created in compliance with AMA guidelines for ethical business practices and are preferably approved by the Pharmaceutical company sponsor.

An interactive detail is preferably designed to engage the physician. For example, physicians are preferably prompted to answer strategically placed questions throughout the detail. These questions can serve to increase recall, create positive product usage behavior change, and provide marketing teams with valuable marketing feedback. The answers to the questions are preferably logged by the data accumulation module 206 and are then reported to the sponsoring pharmaceutical company by the reporting module 208. The interactive details are preferably designed around templates that enable content to be effectively moved in and out of the presentations.

The system preferably also records time spent on each part of the detail and allows users to bring up supporting information through a roll over (mouse over) system of pop up windows. Information requests from the user are preferably handled through designed forms that are populated with information from the database about the doctor. Some details can use tree logic wherein the doctor answers a series of questions and in response is sent to appropriate types of interactive presentations. Interactive details can have interactive mechanisms of action that allow the user to drag and drop pieces of a puzzle into a scene in order to compete the action. The user actions in response to these interactive mechanisms are preferably recorded, stored, and presented real-time by the data accumulation module 206, the data storage module 207, and the reporting module 208.

An example of a question and answer session in a detail is given below:

Please answer the following question to continue:

Naftin provides a >90% cure or global improvement rate in
both tinea cruris and tinea pedis
True
False

A mechanism of action (MOA) replication feature teaches and/or reinforces the users understanding of a drug's MOA. In accordance with one embodiment, the MOA replication feature requires the user to drag a medication with the mouse to a graphic representation of the MOA and to place the product where the user believes it belongs. In essence, the user can be required to clearly understand where a medication acts within the cell or system.

The content delivered in the detail is preferably pre-approved marketing material received directly from the pharmaceutical company. A detail is preferably also approved by a sponsoring pharmaceutical company. This material is preferably transformed into HTML and/or Flash format and is sized to download via a 28.8 Kbps modem.

An interactive detail preferably verifies that the user has completed the required screens and also verifies completion of the detail.

The hosting module 204 preferably detects the browser type and the flash capability of each user. The hosting module preferably automatically sends the users to the HTML version of the detail if the browser type is not compatible with the Flash plug in or does not have the Flash plug in.

C. Data Accumulation and Data Storage Modules

The data accumulation module 206 preferably captures data supplied by and captured from the user while using the web site 108. The data can include, for example, URLs accessed and associated timestamps, responses to interactive details, personal or contact information for physicians, and logs of a physician's use of the system (e.g., pages accessed and progress through each detail).

Some accumulated data can be accumulated for the use of all drug companies 104 that use the system 102, such as, for example, the identities of the system's users. Other data can be accumulated for the specific use of certain drug companies, such as, for example, responses to interactive details, which can be accumulated for the use of a drug company sponsoring a detail.

The data accumulation module 206 preferably stores accumulated data on the data storage module 208. The data captured from user interaction with the system 102 is preferably stored in a database designed for ease of report generation and for facilitation of rapid verification when a user returns to the web site 108 for future interaction. Most of the database access is via stored procedures. This provides for fast response times for all major data accumulation operations. For example, a system of data capture and storage tracks different ways a physician enters the site and allows this information to be presented in a format that can be used to evaluate recruiting methods.

If a physician is recruited by multiple means (e.g., email, fax, direct mail), different user names and passwords can be provided to the physician or assigned to the physician for each recruiting method. The system 102 preferably differentiates between different recruiting methods using the username supplied for that method. All physicians that are targeted by the same recruiting method can be given the same username but different passwords. This technique enables the system 102 to report which methods are effective, and enables the system 102 to compile and analyze various recruiting methods for a particular physician over a series of interactive detail programs. The effectiveness information can be customized and presented through the reporting module 208.

The data storage module preferably utilizes a Microsoft's SQL Server relational database management system. The database is preferably stored on a RAID 5 disk array, while the database servers are preferably hosted on a clustered Windows 2000 system containing two database servers. The two database servers provide redundancy and enable failover capability such that that if one database server goes down, another server takes on the load automatically and processes database requests.

D. Reporting Module

The reporting module 208 preferably provides comprehensive reporting to drug companies on a real-time basis. A company or its representative can log in to a web site provided by the system to view reports. A specific URL can be provided for each drug company that uses the system and/or each drug involved, Real time reports are preferably made available 24 hours a day, 7 days a week (i.e., 24×7).

The reporting module 208 preferably displays the user information collected from the authentication module 202, the hosting module 204, and the marketing module 216 to track the progress of each user and the success of the recruitment.

In accordance with one embodiment, reports can include, for example, the following content:

Number of physicians detailed

Names of physicians detailed

Professional information (Physician specialty, sub-specialty, ME number)

Time spent on detail (Start Time/ Stop Time/ Total Time)

Total questions answered on interactive portion

Questions answered correctly/incorrectly

Physician's comments (if any)

Physician “click through” activity for more information (web sites, product sites, studies, etc.)

The reporting module 208 preferably only identifies users who have completed the authentication module 202 and have accessed the hosting module 204 to start an interactive detail. A user who has abandoned the process prior to this point is preferably not considered a responder and is not counted on a report. Those users who are not automatically verified in the authentication module 202 can be included in the reports and identified as “Awaiting Manual Verification” in the reporting module 208.

E. Customer Relationship Management Integration Module

The customer relationship management (CRM) integration module 210 is preferably configured to interface with customer relationship management systems of one or more drug companies. The CRM integration module 210 functions to integrate data collected by the system 102 into the customer relationship management system of a drug company. This integration enables the drug company to use its own CRM system to access data collected by the system 102. The information to be integrated can include, for example, physician activity on the web site 108, virtual details viewed, and questions answered (such as during an interactive detail).

In accordance with one embodiment, the CRM integration module 210 supports the downloading of data by a drug company from the web site 108 or through another mechanism. For example, the data can be configured in the format of a spreadsheet or in XML format.

In one embodiment, a direct communication link is established between a drug company CRM system and the CRM integration module 210 to enable information to be transmitted to the CRM system in real-time.

The CRM integration module 210 preferably integrates data collected in the system 102 into the customer relationship management system, sales force automation, or call center, of the pharmaceutical company. This information can supplement the data currently in the pharmaceutical company's system. The information can include, for example, physician name and address, web site activity, virtual details viewed, questions answered, information requested, samples requested, and rep visits requested.

The CRM integration module 210 represents a significant level of collaboration between the system 102 and the pharmaceutical company. The creation of a real time data link into the pharmaceutical company system requires close coordination between the two companies' information technologies departments to align each table and data element. Therefore, the specific data fields and configuration are particular to each implementation. The specific data fields and configuration can be used to facilitate one-to-one marketing programs by providing real-time personalized data to pharmaceutical company CRM applications. Thus, the CRM integration module enables pharmaceutical customers to develop and target their marketing materials and promotional message according to specific, personalized physician demographic and/or prescribing data.

F. Representative Access Module

The representative access module 212 provides pharmaceutical companies with unique sales force enhancement tools to leverage current investments in sales force infrastructure. In accordance with one method, representatives can extend their presence beyond a sales call by inviting a busy physician to view an interactive detail through the system 102 at the physician's convenience, 24 hours a day, from home or office.

A method of inviting a physician to view interactive details enables the system 102 to ensure that it is allowing viewing by only those users that are targeted by a sales representative. The system 102 can rely upon, for example, a unique user name and password for verification of an invitation. The method of invitation and verification enables timely presentation of essential marketing material in a controlled system.

The access module 212 can also host home pages for sales representatives to allow targeted physicians to directly and conveniently access and communicate with representatives via the Internet. The home pages can be configured, for example, to enable physicians to:

request samples

request an appointment

request product information

contact the representative, through e-mail.

G. Call Center Module

The call center module 214 preferably enables drug companies to provide exemplary customer service for targeted physicians. Physicians routinely contact call centers at pharmaceutical companies for customer service needs such as requesting product samples, requesting patient education material, requesting off-label product information, reporting adverse events, requesting free medication for indigent patients, and the like. Research has demonstrated the following results with respect to the customer service needs of physicians:

75% of Internet using physicians are interested in ordering samples online

27% of physicians have contacted a drug company call center at least once within the last year and 23% have made 3 to 5 contacts

49% of physicians reported that they do not have the appropriate contact information when the need for customer service arises.

Call centers of drug companies typically operate during standard business hours, and very few of these centers are equipped to provide customer service via the Internet.

The call center module 214 is preferably configured to support:

Sample requests

Representative appointments

Patient assistance program forms

Patient education

Off-label product information

Clinical trial updates

Adverse event reporting

The call center module 214 is preferably configured to be accessible 24 hours a day via a toll-free phone number, via e-mail, via web-based chat, or via a combination of channels.

The call center module 214 enables direct interaction by physicians with dozens of companies through request submissions that can be populated with information from users' profiles in the data storage module 207. The system 102 preferably routes each request to a company at a predetermined fulfillment organization.

H. Marketing Module

The marketing module 216 preferably supports recruitment and segmentation activities. The marketing module 216 preferably compares a pharmaceutical company invited user list with a member database (e.g., stored by the storage module 207) and segments out the physicians recruited by email and direct mail. The marketing module 216 is preferably also used with manual verification to check the user against the invited user list, in addition to the AMA database. The marketing module 216 preferably provides marketing campaign management and assists the system 102 and the drug companies 104 with recruiting and message development.

Business rules are preferably applied to build recruiting plans by segmenting the user database. In addition to physician name and specialty, the system 102 can determine other contact information for the physicians and can place each physician into a segment within the recruiting plan. Once physicians respond to the plan, the marketing module 216 can record the channel that was responsible for recruiting the physician and determine the acquisition costs for the campaign.

Data from previous detailing programs is preferably collected and sorted to determine which messages are effective and which messages are not effective. This data can then be used to reshape future campaigns.

The marketing module 216 can also operate on syndicated prescription data for each physician for the purposes of return on investment tracking.

I. Honorarium Module

The honorarium module 218 preferably provides an honorarium or gift via email to physicians upon completion of the detail and authentication. The system 102 provides honoraria (gifts) in response to physicians' completion of interactive details. In one embodiment, the honoriaria can be offered only to certain targeted physicians. Alternatively, the honoriaria can be offered to all physicians. In one embodiment, physicians to whom honoraria are to be offered are invited to access the web site, to participate in an interactive detail, and to receive an honorarium. Invitations can be transmitted, for example, by e-mail.

In one embodiment, each user is assigned an honorarium code from a contracted vendor, e.g., Amazon.com, which is embedded into a link. The user clicks on the link and is automatically taken to the appropriate fulfillment page on the vendor's web site.

In one embodiment, a physician who has actively viewed a number of presentations can use accumulated honoraria to purchase books, videos, or the like from a contracted vendor.

III. Methods

The physician authentication module 202 preferably interacts with the interactive detail hosting module 204, thereby ensuring that only verified users gain access to interactive details. The physician authentication module 202 preferably also interacts with the data accumulation module 206 by logging verification information entered by the user. In accordance with one embodiment, the information provided by the user through the authentication module 202 is stored, for example, by the data accumulation module 206, in the data storage module 207.

In accordance with one embodiment, the system 102 enables users to request assistance from pharmaceutical companies. The information accumulated in the data storage module 207 is used to expeditiously populate specially designed forms with physician personal information that is drawn from the data storage module 207. The specially designed forms can be forwarded to pharmaceutical companies to request assistance.

The authentication module 202 preferably interacts with the hosting module 204 and the honorarium module 218 to identify those users who are eligible for an honorarium. Once an invited user completes the authentication module 202, the user may view an interactive detail on the hosting module 204 and will receive an honorarium upon completion via the honorarium module 218.

In one embodiment, if a user is not automatically verified, the user can still view an interactive detail, but the user will not receive an honorarium until the user is manually verified. The users flagged for manual verification are preferably stored in a separate database table in the data storage module 207 and must either pass manual verification or provide their licensing information.

The honorarium module 218 preferably uses the information from the authentication module 202 and the hosting module 204 to determine if and where to send the honorarium. If the user completes required fields, screens, and questions in an interactive detail, the honorarium module 218 preferably sends an honorarium notification to the user name and email address given in the authentication module 202.

The user data from the authentication module 202 is preferably stored in the data storage module 206 and is made available for transfer to the marketing module 216 for use in future recruitment programs. The user name and address is also preferably made available for transfer to the CRM module 210, to the representative access module 212, and to the call center module 214 for follow up activities by a pharmaceutical company.

The reporting module 208 preferably only records those physicians who have been authenticated by the authentication module 202 and have started participation in an interactive detail hosted by the hosting module 204. A user who has abandoned the process prior to beginning an interactive detail is preferably not considered a responder and is not counted on a report. Those users who are not automatically verified in the authentication module 202 can be included in reports and can be identified as Awaiting Manual Verification by the reporting module 208.

The data storage module 207 preferably stores the data accumulated by the data accumulation module 206. The data accumulation module 206 preferably accumulates and stores on the data storage module 207 all of the data received by the authentication module 202, the hosting module 204 and the honorarium 218 module.

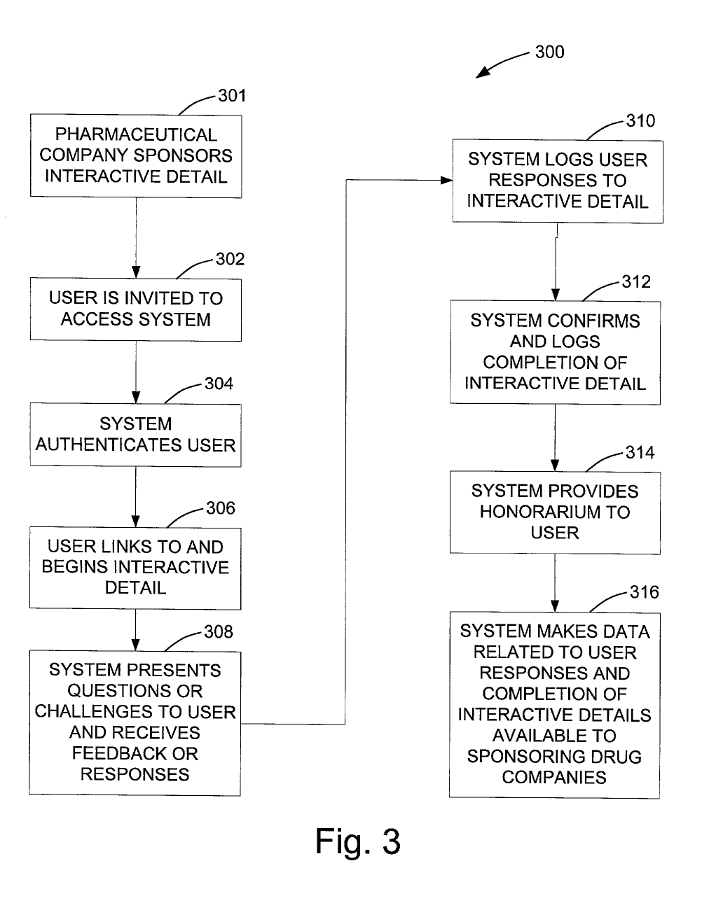

FIG. 3 illustrates a method 300 in accordance with one embodiment of the invention. As will be understood by one skilled in the art, other methods and variations of the method 300 are also within the scope of the invention.

At a step 301 a pharmaceutical company 104 agrees to sponsor the hosting of an interactive detail through the system 102. The sponsorship of the interactive detail preferably includes payment in exchange for a service of hosting and presenting the interactive detail, as well as providing data accumulated in conjunction with the presentation of the interactive detail to the pharmaceutical company.

At a step 302 a user (physician) 106 is invited to access the system 102 through the web site 108. The invitation can be effected, for example, through an e-mail, an advertisement, a URL on a representative's business card.

At a step 304, the system 102 authenticates the user. In one embodiment, the user supplies authentication information, such as is described in Section II.A above. Alternatively, a user can be supplied a login name and password on a first access and the user can reuse the login information for subsequent site accesses.

At a step 306, the user links to and begins interactive detail. The interactive detail can be reached in various ways, such as through hypertext links from any location on the web site 108 or elsewhere. The interactive detail is preferably presented in accordance with Section II.B above.

At a step 308, the system presents questions or challenges to the user and receives user feedback or responses. The questions or challenges can be presented as described in Section II.B above. One or more questions of each interactive detail preferably relate to the mechanism of action of a prescription drug.

At a step 310, the system preferably logs or records user responses to interactive details. The system preferably accumulates and stores the responses as described in Section II.C above.

At a step 312, the system preferably confirms the user's completion of an interactive detail. Completion of an interactive detail may be configured to confirm the user's understanding of the concepts presented in the interactive detail. For example, an interactive detail can be configured to re-ask questions until a user has demonstrated sufficient understanding before the interactive detail completes.

At an optional step 314, the system provides an honorarium to the user in response to the successful completion of an interactive detail. The honorarium can be effected in accordance with Section II.I above.

At a step 316 the system makes data related to users responses to and completions of interactive details available to drug companies 104 sponsoring the details. The step 316 can be effected in accordance with the Section II.D above.

IV. Conclusion

Although the invention has been described in terms of certain embodiments, other embodiments that will be apparent to those of ordinary skill in the art, including embodiments which do not provide all of the features and advantages set forth herein, are also within the scope of this invention. Accordingly, the scope of the invention is defined by the claims that follow. In method claims, reference characters are used for convenience of description only, and do not indicate a particular order for performing a method.

What is claimed is:

1. A system for providing pharmaceutical information to physicians, the system comprising:
a physician authentication module configured to authenticate that a user is a registered physician;
a presentation hosting module configured to present a plurality of interactive presentations to users that have been authenticated by the physician authentication module, wherein each presentation is related to a prescription drug; and
a data accumulation module configured to accumulate user responses to the interactive presentations.

2. The system of claim 1, further comprising
a presentation storage module storing the plurality of interactive presentations.

3. The system of claim 1, further comprising
an honorarium module configured to provide honoraria to users to whom interactive presentations have been presented.

4. The system of claim 3, wherein the honorarium module is further configured to provide honoraria only to selected users to whom interactive presentations have been presented.

5. The system of claim 3, wherein the honorarium module is configured to verify that a user has responded to questions presented in one of the interactive presentations before providing an honorarium.

6. The system of claim 3, wherein the honorarium module is further configured to verify that a user has completed participation in one of the interactive presentations before providing an honorarium

7. The system of claim 1, further comprising
a reporting module configured to provide data related to users that have viewed presentations.

8. The system of claim 7, wherein the data related to users that have viewed presentations comprise a number of users to which a presentation has been presented.

9. The system of claim 7, wherein the data related to users that have viewed presentations comprise names of users to which a presentation has been presented.

10. The system of claim 7, wherein the data related to users that have viewed presentations comprise responses by the users to the presentations.

11. A method for providing information related to pharmaceuticals to physicians, the method comprising:
(a) inviting a user to access a system configured to provide the information related to pharmaceuticals;
(b) through the system, authenticating that the user is a physician;
(c) through the system, presenting information related to a pharmaceutical to the user;
(d) through the system, prompting the user to provide input confirming the user's comprehension of at least a portion of the presented information; and
(e) through the system, receiving input provided by the user in response to (d).

12. The method of claim 11, further comprising through the system, providing an honorarium to the user in response to at least (e).

13. The method of claim 12, wherein the honorarium is a credit that can be redeemed at an on-line vendor.

14. The method of claim 12, wherein the honorarium is provided only to targeted users.

15. The method of claim 11, further comprising
receiving value from a pharmaceutical company in exchange for performing at least (b), (c), (d), and (e).

16. The method of claim 11, further comprising
performing (a), (b), (c), (d), and (e) with respect to a plurality of users; and
accumulating input provided by the plurality of users in response to (e).

17. The method of claim 16, further comprising
maintaining statistics relating to accumulated input provided by the plurality of users.

18. The method of claim 17, further comprising
providing the statistics to at least one pharmaceutical company.

19. A method comprising hosting interactive presentations related to prescription pharmaceuticals, accumulating user responses to the interactive presentations, presenting data related to accumulated user responses to drug companies sponsoring the interactive presentations, and providing honoraria to users in exchange for participation in the interactive presentations.
